(19) United States
(12) Patent Application Publication
(10) Pub. No.: US 2010/0248352 A1
(43) Pub. Date: Sep. 30, 2010

(54) SENSOR FOR BIOLOGICAL DETECTION

(75) Inventors: Hyun-Woo Song, Daejeon (KR); Hyeon-Bong Pyo, Daejeon (KR); Yo-Han Choi, Daejeon (KR)

Correspondence Address: LADAS & PARRY LLP, 224 SOUTH MICHIGAN AVENUE, SUITE 1600, CHICAGO, IL, 60604, US

(73) Assignee: ELECTRONICS AND TELECOMMUNICATIONS RESEARCH INSTITUTE, Daejeon (KR)

(21) Appl. No.: 12/746,559

(22) PCT Filed: May 9, 2008
(86) PCT No.: PCT/KR08/02642

§ 371 Date: Jun. 7, 2010

(30) Foreign Application Priority Data
Dec. 17, 2007 (KR) 10-2007-0132363

(51) Int. Cl. C12M 1/34; G01B 9/02; G01N 21/00

(52) U.S. Cl. 435/288.7; 356/450; 356/477; 422/82.05; 422/69; 435/287.2

(57) ABSTRACT

Provided is a sensor for biological detection. The sensor for biological detection includes: a sensing unit including a light generator, an optical coupler, and an optical detector, the optical coupler dividing light incident from the light generator to project divided lights into a bio chip and a reference unit, respectively, and coupling the lights reflected from the respective bio chip and reference unit as one output light, and the optical detector detecting the output light, and the bio chip is independently separated from the sensing unit to be disposed on paths of lights divided by the optical coupler. The sensing unit has a composition of the Michelson interferometer.

### TECHNICAL FIELD

The present invention disclosed herein relates to a sensor for biological detection, and more particularly, to a sensor for biological detection capable of optically measuring a bio signal by using an interferometer.

The present invention has been derived from research undertaken as a part of IT R & D program of the Ministry of Information and Communication and Institution of Information Technology Association (MIC/IITA) [2006-S-007-02], Ubiquitous health monitoring module and system development.

### BACKGROUND ART

Applicable fields and related industries of a bio chip become extensively diversified, for example, environmental pollutant detection and virus detection for environments or foods as well as medical fields such as disease diagnosis, productions of new medicines, and toxic tests.

The bio chip is a hybrid device having a structure of a typical semiconductor chip. The hybrid device is manufactured by combining bio-organic materials with inorganic materials. Herein, the bio-organic materials include enzymes, proteins, antibodies, deoxyribonucleic acids (DNAs), microorganisms, animals and plants cells and organs, nerve cells and organs, and nerve cells, which are originated from a living thing. The inorganic materials include semiconductors and glasses. The bio chip serves as a new functional device for processing new information, which utilizes original functions of bio-molecules and imitates bio-functions to diagnose infective disease or to analyze a gene.

The bio chip may be classified into a DNA chip, a ribonucleic acid (RNA) chip, a protein chip, a cell chip, and a neuron chip according to the degree of systematization and bio-molecules. The bio chip may further extensively include a bio sensor capable of detecting and analyzing various biochemical materials, which is similar to a lab-on-a-chip which is miniaturized and integrated in order to perform automatic analysis functions for sample preparation, biochemical reaction and detection, and data interpretation.

A method of detecting a bio signal includes a method of tagging a bio sample with materials such as a fluorescent material and an enzyme, and a method of using an electrochemical reaction of a bio sample or a surface plasmon resonance (SPR). The method of the tagging the bio sample is to detect an optical signal. Additionally, the method of the tagging the bio sample with the fluorescent material and enzyme may be advantageous to low concentration detection. Because a bio signal typically exists in a low concentration state, the method of the tagging the bio sample with the fluorescent material and enzyme is mainly used.

A sensor for biological detection that optically detects a bio signal may have various methods and structures. The sensor for biological detection may be implemented using a method of directly measuring the intensity of an optical signal occurring from a tag material, and a method of measuring an optical interference signal with an interferometer.

The method of directly measuring the intensity of an optical signal may be a method of directly measuring fluorescence occurring from a fluorescent material or a method of measuring changes in light intensity by a tag material. The method of measuring the optical interference signal may be a method of measuring interference characteristics between lights emitted from a bio sample tagged with a tag material and a reference sample providing a reference value for the bio sample through the Young s interferometer or the Mach-Zehnder interferometer.

The sensor for biological detection measuring the combination of chemical or biological components by using a light intensity change through a tag material includes an optical detector with a first portion and a second portion. The first portion is where a clad of a single mode optical fiber is tapered to gradually decrease to a diameter of a core. The second portion is gradually tapered to increase to an original diameter of the clad. Herein, the single mode optical fiber is a part of an optical waveguide. A recognition part such as silane is attached to the optical detector, in order to allow the recognition part to have combination of chemical or biological components. As a light inputted through an optical input unit passes through the recognition part attached to the optical detector, light intensity changes. This is detected by an optical output unit. That is, the sensor for biological detection can measure the degree of combining chemical and biological components through an intensity difference between an inputted light and an outputted light (refer to U.S. Pat. No. 5,532,493).

Examples of a method of measuring an optical interference signal through an interferometer include a method of measuring the combination of herpes simplex virus type 1 (HSV-1) using the Young s interferometer, and a method of measuring the combination of chemical or biological species using the Mach-Zehnder interferometer (A. Ymeti, et al., Nano Letters vol. 7, pp. 394˜397, 2006 Dec. 29).

A sensor for biological detection measuring virus through the Young s interferometer exhibits very high sensitivity, and also can directly measure viruses in real time. Although the sensor for biological detection is applied to the detecting of the HSV-1, the sensor for biological detection may be applied to general applications. A method of measuring of virus particles measures movements of interference fringes due to interferences of lights from a reference arm and a measurement arm, after fixing a virus to the surface of the measurement arm with respect to the reference arm of an interferometer. The measuring of the virus using the sensor for biological detection may be possible in a very low concentration of about 850 particles/ml, and furthermore may be possible in single virus according to an extrapolation result.

A sensor for biological detection measuring the combination of chemical or biological species using the Mach-Zehnder interferometer is an interferometer including a polymer optical waveguide. The measuring of the chemical or biological species measures changes of an interference signal due to lights outputted from a reference arm and measurement arm by combining the chemical or biological species on the surface of the measurement arm with respect to the reference arm of an interferometer. This sensor for biological detection measures changes of a refractive index for chemical or biological species in a polymer substrate (refer to U.S. Pat. No. 6,429,023).

In addition to the above examples, it is possible to variously constitute sensors for biological detections using an interferometer. Additionally, an interferometer may include an optical fiber or an optical waveguide, and also may include an optical part in bulk form (e.g., a half minor, an objective lens, a beam splitter). Furthermore, the interferometer may be realized using a chip-shaped optical coupler.

The sensor for biological detection using an optical waveguide of the optical fiber as a sensing unit directly fixes a bio sample to a recognition part attached to the sensing unit. Moreover, the sensor for biological detection using an interferometer fixes a bio sample at one arm of the interferometer. Therefore, the sensor for biological detection is disposably used up or requires an additional structure to clean a measured bio sample fixed to the recognizing part or the one arm of the interferometer, thus leading to an increase in cost for measuring a bio sample or manufacturing a sensor for biological detection.

### DISCLOSURE OF INVENTION

### Technical Problem

The present invention provides a sensor for biological detection capable of improving measurement sensitivity of a bio signal while measuring a bio signal through a non-contact method.

### Technical Solution

Embodiments of the present invention provide sensors for biological detection include: a sensing unit including: a light generator; an optical coupler dividing light incident from the light generator to project divided lights into a bio chip and a reference unit, respectively, and coupling lights reflected from the bio chip and the reference unit as one output light; and an optical detector detecting the output light; and the bio chip independent of the sensing unit, and disposed on paths of lights divided by the optical coupler. The sensing unit has a composition of a Michelson interferometer.

In some embodiments, the optical detector measures a phase change at the bio chip from the output light.

In other embodiments, chemical or biological reactions occur in the bio chip.

In still other embodiments, the bio chip is disposable.

In even other embodiments, the optical coupler is an optical branching/coupling unit including an optical waveguide.

In yet other embodiments, the sensors for biological detection further include a terminal disposed between the optical branching/coupling unit and the bio chip.

In further embodiments, the terminal includes one of a gradient index (GRIN) lens, a micro lens, and a C-type lens.

In still further embodiments, the optical coupler includes a half mirror.

In even further embodiments, a light incident from the light generator is a planar light.

In yet further embodiments, the optical coupler is a chip-shaped vertical coupler.

In yet further embodiments, the reference unit is physically coupled to the sensing unit.

In yet further embodiments, the reference unit is physically coupled to the bio chip.

### Advantageous Effects

As described above, according to the present invention, provided is a sensor for biological detection capable of improving measurement sensitivity of a bio signal by measuring chemical or biological reactions in a bio chip through changes of an interference light intensity.

Additionally, according to the present invention, costs for measuring a bio sample or/and for manufacturing a sensor for biological detection can be reduced because a bio chip is separated from a sensing unit.

### BRIEF DESCRIPTION OF THE DRAWINGS

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIGS. 1 through 3 are conceptual sectional views illustrating a specific reaction occurring in a bio chip;

FIGS. 4 and 5 are graphs measuring changes of absorption spectrum and a refractive index with respect to specific reaction occurring in a bio chip;

FIGS. 6 through 8 are conceptual sectional views and a conceptual perspective view according to embodiments of the present invention; and

FIG. 9 is a graph illustrating changes of a refractive index with respect to specific reaction occurring in a bio chip as changes of interference light intensity according to an embodiment of the present invention.

### BEST MODE FOR CARRYING OUT THE INVENTION

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. In the figures, the dimensions of layers and regions are exaggerated for clarity of illustration. It will also be understood that when a layer (or film) is referred to as being on another layer or substrate, it can be directly on the other layer or substrate, or intervening layers may also be present. Further, it will be understood that when a layer is referred to as being under another layer, it can be directly under, and one or more intervening layers may also be present. In addition, it will also be understood that when a layer is referred to as being between two layers, it can be the only layer between the two layers, or one or more intervening layers may also be present. Like reference numerals refer to like elements throughout.

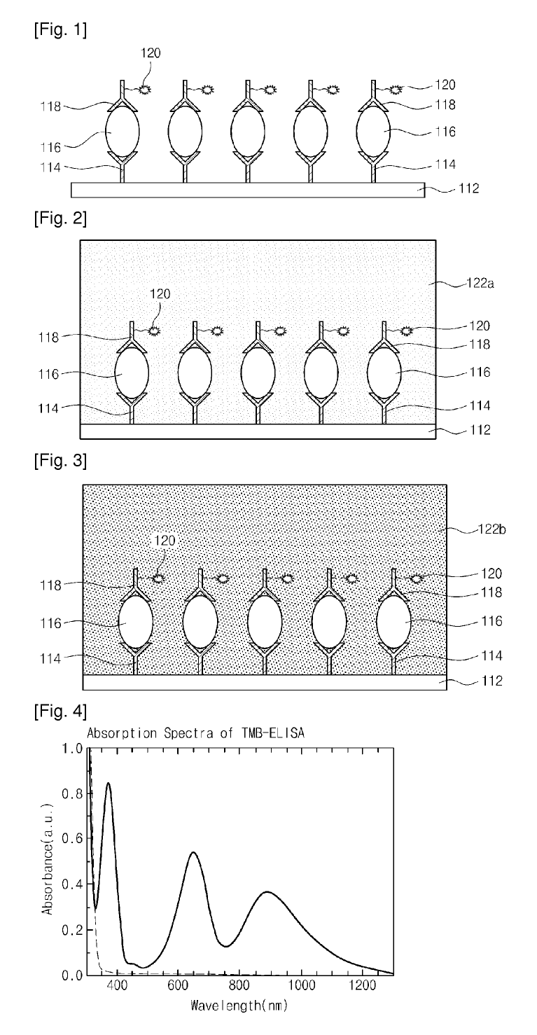

FIGS. 1 through 3 are conceptual sectional views illustrating a specific reaction occurring in a bio chip. To analyze prostate-specific antigen (PSA) protein, provided is an enzyme-linked immunosorbent assay (ELISA) method using a 3,3,5,5-tetramethylbenzidine (TMB) substrate.

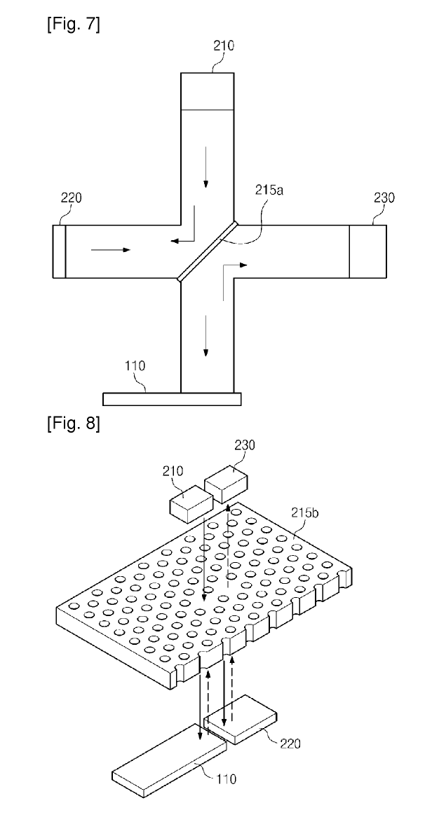

Referring to FIG. 1, first antibodies 114 may be fixed on a reaction part 112 in a bio chip 110 of FIG. 6. A substrate (not shown) of the bio chip may be a crown glass (BK7). A titanium (Ti) layer and a gold (Au) layer with a predetermined thickness may be sequentially deposited and stacked on a predetermined region of the bio chip to form the reaction part 112. The first antibodies 114 may be anti-PSA antibodies.

Antigens 116 may be provided to the reaction part 112 on which the first antibodies 114 are fixed. By means of an immune reaction between the antigens 116 and the first antibodies 114, immune-complexes may be formed, where the antigens 116 are combined to the first antibodies 114.

Second antibodies 118 where enzymes 120 are connected may be provided to the reaction part 112 having the fixed immune-complexes. The second antibodies 118 may be anti-PSA antibodies. By means of an immune reaction between the second antibodies 118 and the antigens 116, the second antibodies 118 may be combined to the antigens 116 of the immuno-complexes.

Referring to FIGS. 2 and 3, a substrate 122a may be provided to the reaction part 112 in the bio chip in which the second antibodies 118 with the enzymes 120 are fixed to the immune-complexes. The substrate 122a may be a TMP substrate. By means of an enzyme reaction between the enzymes 120 and the substrate 122a, the substrate 122a may be converted into a chromogenic substrate 122b. The chromogenic substrate 122b may have a specific color within a visible light range. When the TMB substrate is used as the substrate 122a, the chromogenic substrate 122b may have a blue color. That is, the chromogenic substrate 122b converted by means of an enzyme reaction between the enzymes 120 and the TMB substrate may have distinctive absorption for light having a wavelength of about 652 nm.

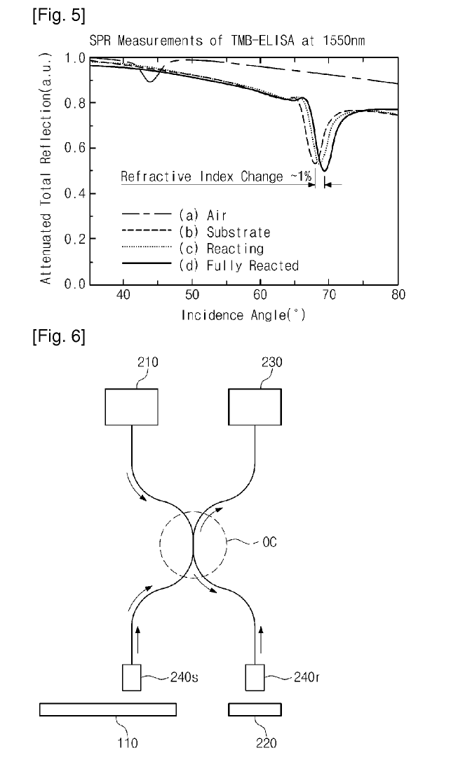

FIGS. 4 and 5 are graphs measuring changes of absorption spectrum and a refractive index with respect to specific reaction occurring in a bio chip.

Referring to FIG. 4, absorption spectrums are shown, which are measured for recognizing characteristics of reaction between the enzyme and a TMB substrate. In the graph, a dotted line represents a state of before converting into a chromogenic TMB substrate, i.e., before the enzyme reacts with the TMB substrate. A solid line represents absorption spectrum after converting into the chromogenic TMB substrate, i.e., after the enzyme reacts with the TMB substrate.

From the solid line of the graph, it can be observed that distinctive absorption occurs with respect to light having a wavelength of about 652 nm. This is because the substrate used in the enzyme reaction is the TMB substrate. The measured absorption spectrum is about an optical path of about 2 nm. As illustrated in the graph, dozens of % of a light absorption signal is obtained with respect to the optical path of about 2 nm. About 53% of a light absorption signal is obtained respect to light having a wavelength of about 652 nm.

Referring to FIG. 5, changes in refractive index are shown, which are measured using surface plasmon resonance (SPR) in order to recognize characteristics of reaction between the enzyme and the TMB substrate. In the graph, a crown glass is used for a substrate of a bio chip, when the first antibodies 114 of FIG. 1 fixed at the reaction part 112 of FIG. 1, the antigens 116 of FIG. 1 coupled to the first antibodies, and the second antibodies 118 of FIG. 1 coupled to the enzymes 120 of FIG. 1 are provided. A titanium layer of an about 2 nm thickness and a gold layer of an about 35 nm thickness are sequentially deposited and stacked to form the reaction part. Line (a) represents a state of before introducing the TMB substrate. Line (b) represents a state of before the enzyme reacts with the TMB substrate after introducing the TMB substrate. Line (c) represents a state of when a small amount of the enzyme reacts with the TMB substrate after introducing the TMB substrate. Line (d) represents changes of each refractive index measured after a large amount of the enzyme and the TMB substrate react with each other in a saturated reaction.

It can be appreciated that a refractive index measured after the enzyme fully reacts with the TMB substrate changes by about 1% from a refractive index measured before the enzyme reacts with the TMB substrate. The change in a refractive index by means of reaction between the enzyme and the TMB substrate can be sensed using an interferometer that measures a phase difference. According to an embodiment of the present invention, the change of the refractive index by the reaction between the enzyme and the TMB substrate are measured using a Michelson interferometer.

FIGS. 6 through 8 are conceptual sectional views and a conceptual perspective view according to embodiments of the present invention.

Referring to FIG. 6, a sensor for biological detection including a composition of a Michelson interferometer with an optical waveguide may be provided. The sensor for biological detection may include a sensing unit and a bio chip 110. The sensing unit may include a light generator 210, an optical coupler OC, and an optical detector 230. The sensor for biological detection may further include a reference unit 220.

The light generator 210 may allow light to be incident to the optical coupler OC through the optical waveguide (or, an optical fiber). The optical coupler OC may divide the light incident from the light generator 210 and may project divided lights toward the bio chip 110 and the reference unit 220 through the optical waveguide. Additionally, the optical coupler OC may couple inflowing lights reflected from the bio chip 110 and the reference unit 220 into one output light. The optical coupler OC may be an optical branching/coupling unit using the optical waveguide.

Chemical or biological reactions may occur in the bio chip 110. The reference unit 220 may provide a reference value for the chemical or biological reactions occurring in the bio chip 110.

The bio chip 110 and the reference unit 220 may be respectively disposed on paths of lights divided by the optical coupler OC. The bio chip 110 may be separated from the sensing unit. Accordingly, it may further include terminals 240s and 240r disposed between the optical coupler OC, the bio chip 110, and the reference unit 220. The terminals 240s and 240r may be used for allowing the divided lights to be projected toward the bio chip 110 and the reference unit 220, and the reflected lights from the bio chip 110 and the reference unit 220 to inflow the optical coupler OC. The terminals 240s and 240r may be one of a gradient index (GRIN) lens, a micro lens, and a C-type lens.

Because the bio chip 110 is separated from the sensing unit, it is disposable. The reference unit 220 may be coupled to the sensing unit. Unlike this, the reference unit 220 may be coupled to the bio chip 110. Accordingly, the sensor for biological detection of the present invention has the bio chip 110 separated from the sensing unit, such that a bio signal can be measured through a non-contact method. That is, while a conventional sensor for biological detection that directly fixes a bio sample to one arm of an interferometer is disposable or requires an additional component for cleansing the measured bio sample, the sensor for biological detection of the present invention may include the disposable bio chip 110. Therefore, cost for measuring a bio sample or/and for manufacturing a sensor for biological detection may be reduced.

The optical detector 230 may detect one output light coupled by the optical coupler OC. The optical detector 230 may measure an interference light intensity of the output light. Accordingly, the sensor for biological detection may measure changes of an interference signal by using interference of lights reflected from the bio chip 110 and the reference unit 220.

Referring to FIG. 7, there is a sensor for biological detection including a composition of a Michelson interferometer using a planar light. The sensor for biological detection may include a sensing unit and a bio chip 110. The sensing unit may include a light generator 210, an optical coupler 215a and an optical detector 230. The sensor for biological detection may further include a reference unit 220.

The light generator 210 may allow the planar light to be incident to the optical coupler 215a. The optical coupler 215a may divide the incident parallel light from the light generator 210, and project divided parallel lights toward the bio chip 110 and the reference unit 220, respectively. Additionally, the optical coupler 215a may couple inflowing lights reflected from the bio chip 110 and the reference unit 220 into one output light. The optical coupler 215a may be a half mirror.

Chemical or biological reactions may occur in the bio chip 110. The reference unit 220 may provide a reference value with respect to the chemical or biological reactions occurring in the bio chip 110.

The bio chip 110 and the reference unit 220 may be respectively disposed on paths of the planar lights divided by the optical coupler 215a. The bio chip 110 may be separated from the sensing unit. Because the bio chip 110 has a structure separated from the sensing unit, it may be disposable. Accordingly, the sensor for biological detection of the present invention may include the bio chip 110 separated from the sensing unit, such that it can be measured through a non-contact method. That is, while a conventional sensor for biological detection that directly fixes a bio sample to one arm of an interferometer is disposable or requires an additional component for cleansing the measured bio sample, the sensor for biological detection of the present invention may include a disposable biochip 110. Therefore, costs for measuring a bio sample or/and for manufacturing a sensor for biological detection may be reduced.

The optical detector 230 may detect one output light coupled by the optical coupler 215a. The optical detector 230 may measure an interference light intensity of the output light. Accordingly, the sensor for biological detection may measure changes of an interference signal by means of interference of lights reflected from the bio chip 110 and the reference unit 220.

Referring to FIG. 8, a sensor for biological detection with a composition of a Michelson interferometer using a chip-shaped vertical coupler may be provided. The sensor for biological detection may include a sensing unit and a bio chip 110. The sensing unit may include a light generator 210, an optical coupler 215b, and an optical detector 230. The sensor for biological detection may further include a reference unit 220.

The light generator 210 may allow light to be incident to the optical coupler 215b. The optical coupler 215b may divide the light incident from the light generator 210, and project divided lights toward the bio chip 110 and the reference unit 220. Additionally, the optical coupler 215b may couple inflowing lights reflected from the bio chip 110 and the reference unit 220 into one output light. The optical coupler 215b may be a vertical coupler (Korean Pat. No. 2006-0123995). The vertical coupler may be a substrate with a crystal lattice structure in which a plurality of cylindrical through holes is periodically disposed in a thickness direction. The substrate may include a main crystal lattice defect constituting a main optical waveguide passing light in a thickness direction and a sub crystal lattice defect constituting a sub optical waveguide passing divided lights or coupled lights in a thickness direction by dividing or coupling light of a specific wavelength band among lights passing through the main optical waveguide. By using this chip-shaped coupler, the sensor for biological detection may be manufactured in a small size.

Chemical or biological reactions may occur in the bio chip 110. The reference unit 220 may provide a reference value for the chemical or biological reactions occurring in the bio chip 110.

The bio chip 110 and the reference unit 220 may be respectively disposed on paths of lights divided by the optical coupler 215b. The bio chip 110 may be separated from the sensing unit. Because the bio chip 110 has a structure separated from the detection unit, it may be disposable. Or, the reference unit 220 may be integrated into the sensing unit. Unlike this, the reference unit 220 may be integrated into the bio chip 110. Accordingly, because the sensor for biological detection of the present invention includes the bio chip 110 separated from the sensing unit, a bio signal may be detected through a non-contact method. That is, while a conventional sensor for biological detection that directly fixes a bio sample to one arm of an interferometer is disposable or requires an additional component for cleansing the measured bio sample, the sensor for biological detection of the present invention may include the disposable bio chip 110. Therefore, costs for measuring a bio sample or/and for manufacturing a sensor for biological detection may be reduced.

The optical detector 230 may detect one output light coupled by an optical coupler 215b. The optical detector 230 may measure an interference light intensity of the output light. Accordingly, the sensor for biological detection may measure changes of an interference signal by means of interference of lights reflected from the bio chip 110 and the reference unit 220.

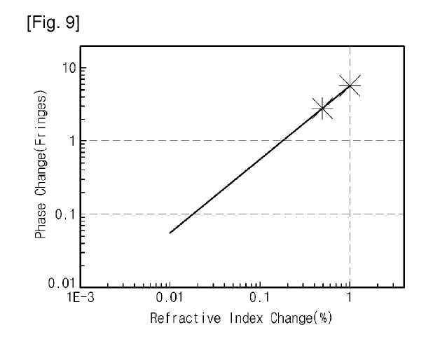

FIG. 9 is a graph illustrating changes of a refractive index with respect to specific reaction occurring in a bio chip as changes of interference light intensity of the present invention.

Referring to FIG. 9, a change period of an interference light intensity is illustrated to identify changes of a refractive index according to the degree of coloring in a TMB substrate through reaction between an enzyme and the TMB substrate occurring in a bio chip. The changes of a refractive index in the TMB substrate is measured by a phase change of an output light. The phase change of the output light is represented by the number of fringes in the output light. The upper asterisk is the number of fringes caused by values that are respectively measured in a saturated reaction when a great amount of the enzymes react with the TMB substrate.

As illustrated in FIG. 5, refractive indexes (an x-axis) have a refractive index change of about 1%, which are respectively measured at a state of before the enzyme and the TMB substrate react with each other and a state of after they completely react with each other. In a case where the change of the refractive index through reaction between the enzyme and the TMB substrate is measured with a phase difference (a y-axis) through an interferometer, a phase difference is measured more than several tens times (about 50 to 60 times).

As illustrated in FIG. 4, although a light absorption signal of an about 53% can be measured by means of reaction between the enzyme and the TMB substrate, a sensor for biological detection using an interferometer of the present invention may measure a relatively high signal with a very small amount of enzyme-substrate reaction. This is because the change of the refractive index with respect to light providing the same level of an interference light intensity is merely about 0.1%. Accordingly, it is apparent that the measurement sensitivity of the sensor for biological detection of the present invention is improved. That is, the sensor for biological detection having greatly improved measurement sensitivity for a bio signal may be provided.

Because the sensor for biological detection of the present invention uses a composition of a Michelson interferometer, changes of an interference light intensity with respect to chemical or biological reactions occurring in a bio chip can be measured. Therefore, the sensor for biological detection having improved measurement sensitivity for the bio signal can be provided.

Additionally, because the sensor for biological detection of the present invention has a bio chip separated from a sensing unit, a bio signal can be measured through a non-contact method. Accordingly, costs for measuring a bio sample or/and for manufacturing a sensor for biological detection can be reduced.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. A sensor for biological detection comprising:
a sensing unit comprising:
a light generator;
an optical coupler dividing light incident from the light generator to project divided lights into a bio chip and a reference unit, respectively, and coupling lights reflected from the bio chip and the reference unit as one output light; and
an optical detector detecting the output light; and
the bio chip independent of the sensing unit, and disposed on paths of lights divided by the optical coupler,
wherein the sensing unit has a composition of a Michelson interferometer.

2. The sensor for biological detection of claim 1, wherein the optical detector measures a phase change at the bio chip from the output light.

3. The sensor for biological detection of claim 1, wherein chemical or biological reactions occur in the bio chip.

4. The sensor for biological detection of claim 1, wherein the bio chip is disposable.

5. The sensor for biological detection of claim 1, wherein the optical coupler is an optical branching/coupling unit including an optical waveguide.

6. The sensor for biological detection of claim 5, further comprising a terminal disposed between the optical branching/coupling unit and the bio chip.

7. The sensor for biological detection of claim 6, wherein the terminal comprises one of a gradient index (GRIN) lens, a micro lens, and a C-type lens.

8. The sensor for biological detection of claim 1, wherein the optical coupler comprises a half minor.

9. The sensor for biological detection of claim 8, wherein a light incident from the light generator is a planar light.

10. The sensor for biological detection of claim 1, wherein the optical coupler is a chip-shaped vertical coupler.

11. The sensor for biological detection of claim 1, wherein the reference unit is physically coupled to the sensing unit.

12. The sensor for biological detection of claim 1, wherein the reference unit is physically coupled to the bio chip.
